(19) United States
(12) Patent Application Publication
(10) Pub. No.: US 2022/0252538 A1
(43) Pub. Date: Aug. 11, 2022

(54) PORTABLE DEVICES AND METHODS FOR DETECTING AND IDENTIFYING COMPOUNDS IN A FLUID SAMPLE

(71) Applicant: Pelican Diagnostics Inc., Burlington (CA)

(72) Inventor: Raj Reddy, Burlington (CA)

(21) Appl. No.: 17/203,684

(22) Filed: Mar. 16, 2021

Related U.S. Application Data
(60) Provisional application No. 63/148,377, filed on Feb. 11, 2021.

(51) Int. Cl. G01N 27/327; C12N 15/11; C12N 9/22; G01N 27/48

(52) U.S. Cl. CPC G01N 27/3272; C12N 15/11; C12N 2800/80; G01N 27/48; C12N 2310/20; C12N 9/22

(57) ABSTRACT

This disclosure relates to portable devices for detecting various biomarker compounds, such as antibodies, antigens, nucleic acids (DNA and RNA), fusion proteins, genes, etc. in a clinical specimen such as saliva or blood for the detection of diseases, viruses, and other common pathogens. Also disclosed herein is the detection and identification of one or more diseases using a portable device comprising an electrochemical CRISPR/Cas based biosensor system.

### CROSS-REFERENCE TO RELATED APPLICATIONS

This application claims the benefit of U.S. Provisional Application No. 63/148,377, filed Feb. 11, 2021, the entire contents of which are incorporated herein by reference in their entirety.

### TECHNICAL FIELD

This disclosure relates to the detection and identification of one or more diseases using a portable device comprising an electrochemical clustered regularly interspaced short palindromic repeats (CRISPR) based biosensor system. Individual diseases can be detected by identification of specific compounds, e.g., genes, nucleic acids, fusion proteins, recombinant proteins, antigens, antibodies, etc., in a clinical specimen, such as saliva or blood.

### BACKGROUND OF THE INVENTION

Several assays are currently in use for the detection of various diseases, including, but not limited to immunofluorescence assays, protein microarray assays, reverse transcription loop mediated isothermal amplification assays (RT-LAMP), viral plaque assays, Hemagglutination assays, viral flow cytometry (FCM), quantitative polymerase chain reaction (qRT-PCR), and enzyme linked immunosorbent assays (ELISA). Despite the high sensitivity of these methods, they are not suitable for large scale screening for multiple samples because of the need for large and expensive equipment, intensive sample preparation, and long turnover time limits. Moreover, these methods require skilled personal to perform the assays and are not suitable for point-of-care testing.

Biotechnology plays a potential role in clinical applications, particularly in the development of biosensors for the identification of biomarkers that may be used for the detection of a disease. For example, various immunosensors have been reported for the detection of one or more diseases using different transducers as improved alternatives to traditional assays. Different electrochemical biosensors have been reported that use square wave voltammetry (SWV), differential pulse voltammetry (DPV), electrochemical impedance spectroscopy (EIS), linear sweep voltammetry (LSV), chronoamperometry and cyclic voltammetry (CV).

An increased understanding of critical physiological pathways for disease progression significantly promotes the development of personalized medicine, which is key for the next generation of the healthcare industry. A simple, rapid detection system that can identify morbigenous genes will be of high utility for the design of personalized medicine. Among the advancements of various types of biosensing platforms, the electrochemical biosensing systems have been robustly developed in the last decade, owing to the cost-effectiveness of the transduction system, the time-efficiency and the simplicity of sample processing, providing a potential deployable point-of-care system.

Nucleic acids are a universal signature of biological information. The ability to rapidly detect nucleic acids with high sensitivity and single-base specificity on a portable platform has the potential to revolutionize diagnosis and monitoring for many diseases, provide valuable epidemiological information, and serve as a generalizable scientific tool. Although many methods have been developed for detecting nucleic acids, they inevitably suffer from trade-offs among sensitivity, specificity, simplicity, and speed. As nucleic acid diagnostics become increasingly relevant for a variety of healthcare applications, detection technologies that provide high specificity and sensitivity at low-cost would be of great utility in both clinical and basic research settings.

### SUMMARY OF THE INVENTION

This disclosure relates to portable devices for detecting various biomarker compounds like antibodies, antigens, nucleic acids (DNA and RNA), fusion proteins, genes, etc. in a clinical specimen such as saliva or blood for the detection of diseases, virus and other common pathogens. Also disclosed herein is the detection and identification of one or more diseases using a portable device comprising an electrochemical CRISPR/Cas based biosensor system. The development of an electrochemical biosensor array for virus and/or disease detection offers a unique possibility to create a low-cost and highly sensitive sensor as a point of care method to detect and quantify the compounds in bodily fluids such as saliva or blood information in real-time. The multiarray biosensor described herein that is integrated with a portable device provides real time data acquisition via wireless modules to transmit and/or receive electrical or electrochemical responses on the communication devices. This fully integrated handheld device successfully exhibits a low detection range with a high specificity.

Also disclosed herein are methods and portable devices that comprise a CRISPR/Cas system that may programmatically bind and cleave RNA, thereby service serving as a platform for RNA detection. The methods and devices may be used to capture the unique electrical response initiated by a cleavage caused by the CRISPR/Cas system. In addition, the methods and devices may signify the quantity of viral load present in a sample obtained from a subject via a digital signal. The methods and devices may also be used to identify a target virus and differentiates wild type vs mutated viral genomes. Further, the methods and devices are capable of detecting ultra low concentration of virions (1 copy/μl in less than 15 minutes.

Disclosed herein are portable devices for detecting and identifying one or more biomarker compounds (e.g., nucleic acids, proteins, genes, etc.) in a fluid sample of a subject. The devices include a biosensor array connected to a device; a sensor module disposed in the housing which collects data which detects and identifies one or more nucleic acids; a communication apparatus disposed to the sensor module which electrically transmits the data collected by sensor module; and a battery disposed to the communication apparatus and the sensor module.

In one embodiment, the device further comprises a processing apparatus electronically or wirelessly connected to the communication apparatus, wherein the processing apparatus receives the data regarding the identity of the one or more nucleic acids from the communication apparatus and processes the data to identify a disease based on the one or more identified nucleic acids. In some embodiments, the processing apparatus transmits the identity of the disease detected in the sample from the processing apparatus to a display.

In one embodiment, the device further comprises an amplifier connected to the sensor module, wherein the amplifier amplifies data collected by the sensor module in the housing.

In some embodiments, the fluid sample is any biofluid sample, such as blood, saliva, tears, urine, plasma, etc.

In one embodiment, the biomarker compound is detected using a CRISPR/Cas system. In some embodiments, the CRISPR/Cas system comprises gRNA for S and N protein and/or muted genes, a Cas protein (e.g., Cas9, Cas12, or Cas13), and reporter sequences. In some embodiments, the CRISPR/Cas system is immobilized on screen printed gold electrodes.

In one embodiment, the sensor module collects data regarding more than one biomarker compound. For example, the sensor module may detect two or more of nucleic acids, proteins, and/or genes.

Also disclosed herein is a sensor comprising a counter electrode; one or more multiple working electrodes which include immobilized biological molecules; a reference electrode; and a support on which the electrodes are disposed.

Also disclosed herein are methods for detecting and identifying one or more biomarker compounds in a sample of a subject. The methods include transmitting a collected sample to a biosensor array, wherein the biosensor array is connected to a housing, wherein the housing comprises a sensor module, a communication apparatus and a battery; collecting data regarding the presence of the one or more biomarker compounds with the sensor module; communicating data via the communication apparatus to a processing apparatus; and processing the communicated data to identify the one or more biomarker compounds.

In some embodiments, the sample is a biofluid sample, such as blood, saliva, urine, tears, mucus, plasma, etc.

In some embodiments, the one or more biomarker compounds are selected from the group consisting of nucleic acids, proteins, genes, antibodies, and antigens.

In some embodiments, the methods further comprise identifying the subject as having a disease (e.g., cancer, infection, and/or virus) based on the identified one or more biomarker compounds. In one embodiment, the biomarker compound is a SARS-CoV-2 nucleic acid, and the disease is COVID19. In one embodiment, the biomarker compound is NTRK, and the disease is lung cancer. In another embodiment, the biomarker compound is ptau protein, and the disease is Alzheimer's disease.

Also disclosed herein are handheld portable devices for detecting and identifying one or more nucleic acids in a fluid sample of a subject. The handheld portable device may include a sensor cartridge comprising: a sample well; and one or more disposable biosensors, wherein a biosensor comprises a Cas13a protein or a Cas12a protein, a thiol functionalized reporter sequence selected from the group consisting of RNA, DNA, aptamer, and synthetic nucleotide, and one or more gRNA for SARS-CoV-2 N RNA, SARS-CoV-2 S RNA, or Orflab gene region of SARS-CoV-2; and a hub station comprising: an electrochemical reader; a communication apparatus; and a battery, wherein the hub station receives the sensor cartridge.

In one embodiment, the device further includes a processing apparatus electronically or wirelessly connected to the communication apparatus, wherein the processing apparatus receives data from the communication apparatus and processes the data to identify a disease based on the one or more nucleic acids. The processing apparatus may transmit the identity of the disease detected in the sample from the processing apparatus to a display.

In some embodiments, the Cas13a protein or Cas12a protein, the thiol functionalized reporter sequence, and the one or more gRNA are immobilized on screen printed gold electrodes. In some embodiments, the biosensor further comprises an electrode assembly comprising one counter electrode, four to eight working electrodes, and one reference electrode. In some embodiments, the biosensor further comprises an amplification agent, e.g., T4 RNA ligase, DNA polymerase, RNA polymerase, and/or t4 pnk. In some embodiments, the biosensor further comprises a binding agent, e.g., carbodiimide compounds and/or carboxyl-reactive crosslinker reactive groups.

Also disclosed herein are methods for detecting and identifying one or more nucleic acid compounds in a sample of a subject. The methods may include receiving a collected sample in a sensor cartridge of a portable device, wherein the sensor cartridge comprises one or more biosensors comprising a Cas13a protein or a Cas12a protein; a thiol functionalized reporter sequence selected from the group consisting of RNA, DNA, aptamer, and synthetic nucleotide; and one or more gRNA for SARS-CoV-2 N RNA, SARS-CoV-2 S RNA, or Orflab gene region of SARS-CoV-2; inserting the sensor cartridge into a hub station of the portable device; analyzing the biosensors with methylene blue tagged RNA or DNA reporter sequence which is thiol group functionalized at one terminal using square wave voltammetry; communicating biosensor data to a processing apparatus via a communication apparatus; detecting one or more SARS-CoV-2 nucleic acids in the sample; identifying the sample as being positive for COVID19; and transmitting the COVID19 diagnosis to a user of the device.

In some embodiments, the sample is a blood or saliva sample. In some embodiments, the diagnosis of COVID19 occurs within 2 minutes of receiving the sample.

The above discussed, and many other features and attendant advantages of the present inventions will become better understood by reference to the following detailed description of the invention.

### BRIEF DESCRIPTION OF THE FIGURES

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

The patent or application file contains at least one drawing executed in color. Copies of this patent or patent application publication with color drawings will be provided by the Office upon request and payment of the necessary fee.

FIG. 1 illustrates examples of a portable device described herein which identifies and detects biomarker compounds in a sample from a subject.

FIG. 2 illustrates a process of identifying and detecting one or more biomarker compounds in a sample from a subject.

FIG. 3 shows square-wave voltammetry (SWV) responses for target concentrations range which is one of the working electrodes.

FIG. 4 illustrates an example of a sensor which may be used in the sensor module of a portable device which identifies and detects one or more compounds in a sample from a subject.

FIG. 5 illustrates an example of a sensor with an immobilized biomolecule, which may be used in the sensor module of a portable device. The sensor with the immobilized biomolecule may identify and detect one or more biomarker compounds in a sample from a subject.

FIG. 6 provides a flow chart exemplifying the interaction of various electronic components associated with a portable device described herein.

FIG. 7 illustrates another example of a portable device described herein which identifies and detects biomarker compounds in a sample from a subject.

FIG. 8 provides a schematic exemplifying the use of a CRISPR/Cas system described herein. A sample is obtained and processed and is then mixed with a Cas13a/sgRNA complex. The addition of a target forms a triple complex of sample/Cas13a/sgRNA. The CRISPR-Cas13a system may perform a complementarity dependent cleavage activity, thereby releasing the electrochemical signaling probe from the electrode surface. The electrochemical current outputs is displayed based on the conformation change induced signal change and the CRISPR cleavage induced signal change.

FIG. 9 demonstrates the performance of a CRISPR/Cas13a enhanced E-DNA sensor. A square wave voltammetry (SWV) plot (in response to 100 fM target) shows a baseline without the target (black), addition of target ssDNA (red), CRISPR/Cas9 enhanced result (blue) and CRISPR/Cas13a enhanced result (green).

The following detailed description includes references to the accompanying drawings, which form part of the detailed description. The drawings depict illustrations, in accordance with example embodiments. These example embodiments, which are also referred to herein as “examples,” are described in enough detail to enable those skilled in the art to practice the present subject matter. The example embodiments may be combined, other embodiments may be utilized, or structural, logical, and electrical changes may be made, without departing from the scope of the claimed subject matter. The following detailed description is, therefore, not to be taken in a limiting sense, and the scope is defined by the appended claims and their equivalents.

### DETAILED DESCRIPTION OF THE INVENTION

Disclosed herein are portable devices and methods for diagnosing a disease, such as viral infections, cancers, neurodegenerative disorders, and/or autoimmune or inflammatory disorders, by detecting and identifying one or more biomarkers or compounds in a fluid sample from a subject. The portable devices described herein may be used for point of care testing and may provide testing results in a matter of minutes, and in some instances in under 1 minute. The electrochemical biosensors may show highly robust and selective detection capability of a variety of analytes, with detection limits in the picomolar and femtomolar range.

Illustrated embodiments herein are directed to systems, methods, and devices for detecting and identifying certain compounds or biomarkers, such as antigens, nucleic acids, proteins, genes, or antibody compounds, in a sample specimen of a subject or person in real-time. Further, certain embodiments of the disclosure can be directed to systems, methods, and devices for diagnosis of a subject or person. Technical effects of certain embodiments of the disclosure may include providing diagnosis and treatment for identified health conditions related to the detection and identification of certain biomarkers in a sample specimen of a subject or person in real-time.

Novel sensor technologies, such as nano-compositions with sensing elements, can be combined with mobile communication devices, such as smart phones, and cloud computing to create technical solutions for analysis, diagnosis, and subsequent treatment of one or more diseases or disorders. Novel sensors used in combination with the processor of a smart phone and/or remote server, and a biomarker processing module or engine with a neural network or pattern matching algorithm, can be used to detect one or more biomarkers from one or more sample specimens. Embodiments of the disclosure can have many useful and valuable applications in the biomedical industries, health care and medical care sectors.

As used herein, “target biomolecule” or “biomarker” refers to a compound associated with a disease or disorder. The signal and/or signal patterns of the target biomolecule are associated with the presence of one or more substances, concentrations and/or amounts of respective substances associated with diagnosing, treating, or addressing a health condition or disease. In some instances, the one or more signals and/or signal patterns associated with a presence of a specific combination of substances at predefined concentrations and/or amounts.

As used herein, “real-time” refers to an event or a sequence of acts, such as those executed by a computer processor that are perceivable by a subject, person, user, or observer at substantially the same time that the event is occurring or that the acts are being performed. By way of example, if a neural network receives an input based on the sensing and identification of a biomarker, a result can be generated at substantially the same time that the biomarker was sensed and identified. The real-time processing of the input by the neural network may have a slight time delay associated with converting the sensed compound to an electrical signal for an input to the neural network; however, any such delay may typically be less than 1 minute and usually no more than a few seconds. In certain embodiments, the analysis of a sample, e.g., receipt of the sample to receipt of the processing results, takes 1 minute to 10 minutes, or in some embodiments, takes less 10 minutes, 9 minutes, 8 minutes, 7 minutes, 6 minutes, 5 minutes, 4 minutes, 3 minutes, 2 minutes, or 1 minute. In some aspects, data acquisition takes less than 1 minute, and in certain aspects, takes about 25 seconds.

Diseases or disorders that can be detected by certain embodiments of the disclosure include viral or bacterial infections, cancers, neurodegenerative disorders, and/or autoimmune or inflammatory disorders.

As used herein, the term “viral infection” refers to an infection caused by the presence of a virus in the body. The virus may be a DNA virus, an RNA virus, or a retrovirus. In some embodiments, a particular viral strain may be identified, including viral strains that differ by a single nucleotide polymorphism Examples of viral infections which can be detected by certain embodiments of the disclosure can include, but are not limited to, adenovirus, enterovirus, human coronavirus, human metapneumovirus, rhinovirus (RV), influenza, (e.g., influenza A or influenza B), parainfluenza and respiratory syncytial virus (RSV), severe acute respiratory syndrome (SARS), severe acute respiratory syndrome coronavirus (SARS-CoV), Middle East respiratory syndrome (MERS), Middle East respiratory syndrome coronavirus (MERS-CoV), coronavirus, severe acute respiratory syndrome coronavirus 2 (SARS-CoV-2), adeno-associated virus, aichi virus, banna virus, bunyamwera virus, bunyavirus, cercopithecine herpesvirus, chandipura virus, chikungunya virus, dengue virus, dhori virus, dugbe virus, duvenhage virus, eastern equine encephalitis virus, ebolavirus, measels virus, echovirus, encephalomyocarditis virus, GB virus C/hepatitis G virus, hantaan virus, hepatitis (e.g., hepatitis A, hepatitis B, or hepatitis C), human immunodeficiency virus (HIV) (e.g., HIV1 or HIV2), lassa virus, lymphocytic choriomeningitis virus, marburg virus, mayaro virus, measles virus, mengo encephalomyocarditis virus, mokola virus, nipah virus, rabies virus, rhabdovirus, rosavirus, rotavirus, rubella virus, salivirus, sapporo virus, seoul virus, variola virus, yellow fever virus, and zika virus.

In some aspects, the virus may be selected from the group consisting of human respiratory syncytial virus, Sudan ebolavirus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyoxivirus, Australian bat lyssavirus, Avian bornavirus, Avian metapneumovirus, Avian paramyoxviruses, penguin or Falkland Islandsvirus, BK polyomavirus, Bagaza virus, Banna virus, Bat hepevirus, Bat sapovirus, Bear Canon mammarenavirus, Beilong virus, Betacoronoavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwere virus, Caliciviridae virus. California encephalitis virus, Candiru virus, Canine distemper virus, Canaine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, DobravaBelgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyoxiviurs SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human gentialassociated circular DNA virus-1, Human herpesvirus 1-8, Human immunodeficiency virus 1/2, Huan mastadenovirus A-G, Human papillomavirus, Human parainfluenza virus 1-4, Human paraechovirus, Human picobirnavirus, Human smacovirus, Ikoma lyssavirus, Ilheus virus, Influenza A-C, Ippy mammarenavirus, Irkut virus, J-virus, JC polyomavirus, Japanses encephalitis virus, Junin mammarenavirus, KI polyomavirus, Kadipiro virus, Kamiti River virus, Kedougou virus, Khuj and virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2Y225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobalamammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O′nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Procine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, and Zygosaccharomyces bailii virus Z (ZbV-Z).

In some embodiments, a virus (e.g., an RNA virus) is selected from the group consisting of Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, and a Deltavirus. In certain embodiments, a virus is selected from the group consisting of Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In some embodiments (e.g., a DNA virus) is selected from the group consisting of Family Myoviridae, Podoviridae, Siphoviridae, Alloherpesviridae, Herpesviridae (including human herpes virus, and Varicella Zorter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, and Rhizidovirus.

As used herein, the term “bacterial infection” refers to an infection caused by the presence of a bacteria in the body. Examples of bacterial infections which can be detected by certain embodiments of the disclosure can include, but are not limited to, Escherichia coli, Klebsiella pneumoniae, Pseudomonas aeruginosa, Staphylococcus aureus, Acinetobacter baumannii, Candida albicans, Enterobacter cloacae, Enterococcus faecalis, Enterococcus faecium, Proteus mirabilis, Staphylococcus agalactiae, and Staphylococcus maltophilia.

As used herein, the term “cancer” refers to a disorder in which a population of cells has become, in varying degrees, unresponsive to the control mechanisms that normally govern proliferation and differentiation. Cancer refers to various types of malignant neoplasms and tumors, including metastasis to different sites. Examples of cancers which can be detected by certain embodiments of the disclosure can include, but are not limited to, brain, ovarian, colon, prostate, kidney, bladder, breast, lung, oral, and skin cancers. Specific examples of cancers are: adenocarcinoma, adrenal gland tumor, ameloblastoma, anaplastic tumor, anaplastic carcinoma of the thyroid cell, angiofibroma, angioma, angiosarcoma, apudoma, argentaffinoma, arrhenoblastoma, ascites tumor cell, ascitic tumor, astroblastoma, astrocytoma, ataxia-telangiectasia, atrial myxoma, basal cell carcinoma, benign tumor, bone cancer, bone tumor, brainstem glioma, brain tumor, breast cancer, vaginal tumor, Burkitt's lymphoma, carcinoma, cerebellar astrocytoma, cervical cancer, cherry angioma, cholangiocarcinoma, a cholangioma, chondroblastoma, chondroma, chondrosarcoma, chorioblastoma, choriocarcinoma, larynx cancer, colon cancer, common acute lymphoblastic leukemia, craniopharyngioma, cystocarcinoma, cystofibroma, cystoma, cytoma, ductal carcinoma in situ, ductal papilloma, dysgerminoma, encephaloma, endometrial carcinoma, endothelioma, ependymoma, epithelioma, erythroleukaemia, Ewing's sarcoma, extra nodal lymphoma, feline sarcoma, fibroadenoma, fibrosarcoma, follicular cancer of the thyroid, ganglioglioma, gastrinoma, glioblastoma multiforme, glioma, gonadoblastoma, haemangioblastoma, haemangioendothelioblastoma, haemangioendothelioma, haemangiopericytoma, haematolymphangioma, haemocytoblastoma, haemocytoma, hairy cell leukaemia, hamartoma, hepatocarcinoma, hepatocellular carcinoma, hepatoma, histoma, Hodgkin's disease, hypernephroma, infiltrating cancer, infiltrating ductal cell carcinoma, insulinoma, juvenile angiofibroma, Kaposi sarcoma, kidney tumor, large cell lymphoma, leukemia, chronic leukemia, acute leukemia, lipoma, liver cancer, liver metastases, Lucke carcinoma, lymphadenoma, lymphangioma, lymphocytic leukaemia, lymphocytic lymphoma, lymphocytoma, lymphoedema, lymphoma, lung cancer, malignant mesothelioma, malignant teratoma, mastocytoma, medulloblastoma, melanoma, meningioma, mesothelioma, metastatic cancer, Morton's neuroma, multiple myeloma, myeloblastoma, myeloid leukemia, myelolipoma, myeloma, myoblastoma, myxoma, nasopharyngeal carcinoma, nephroblastoma, neuroblastoma, neurofibroma, neurofibromatosis, neuroglioma, neuroma, non-Hodgkin's lymphoma, oligodendroglioma, optic glioma, osteochondroma, osteogenic sarcoma, osteosarcoma, ovarian cancer, Paget's disease of the nipple, pancoast tumor, pancreatic cancer, phaeochromocytoma, pheochromocytoma, plasmacytoma, primary brain tumor, progonoma, prolactinoma, renal cell carcinoma, retinoblastoma, rhabdomyosarcoma, rhabdosarcoma, solid tumor, sarcoma, secondary tumor, seminoma, skin cancer, small cell carcinoma, squamous cell carcinoma, strawberry hemangioma, T-cell lymphoma, teratoma, testicular cancer, thymoma, trophoblastic tumor, tumorigenic, vestibular schwannoma, Wilm's tumor, or a combination thereof.

As used herein, the terms an “autoimmune disease” or “inflammatory disorder” refer to a disease caused by the immune system attacking the body's own tissues, resulting in inflammation. Examples of autoimmune diseases or inflammatory disorders which can be detected by certain embodiments of the disclosure can include, but are not limited to, psoriasis, rheumatoid arthritis, Crohn's disease, ulcerative colitis, tuberculosis, lupus, irritable bowel syndrome, and multiple sclerosis.

In some embodiments, the one or more compounds which are detected and identified using the portable devices are detected and identified directly. In some aspects, the one or more compounds are nucleic acids (e.g., RNA or DNA), proteins (e.g., fusion proteins or recombinant proteins), antibodies, and antigens. In certain embodiments, the one or more compounds are nucleic acids. Non-limiting examples of the one or more compounds that may be detected include anti-gp41, anti-gp120, SC2A, IgG, anti-M2, BCN antibodies, monoclonal antibodies (MAbs), anti-CD4bs, ADCVI antibody, anti-HA, anti-FLAG, SARS-CoV-2-N antibody, SARS-CoV-2 N protein, SARS-CoV-2 N RNA, SARS-CoV-2-S antibody, SARS-CoV-2-S protein, SARS-CoV-2 S RNA, SARS-CoV-2 E protein, SARS-CoV-2 E antibody, SARS-CoV-2 N Ab (IgG), Orflab gene region of SARS-CoV-2, spike glycoprotein antibody, p21 antibody, ACE2 antibody, gp150 antibody, CD147 antigen antibody, serine 2 antibody, MERS-CoV spike (S) protein, anti-HA antibody, anti-rhinovirus antibody, anti-HCV antibodies, HCoV-229E antigen protein, prostate specific antigens, influenza A antigens, influenza B antigens, IL6, ptau181, NTRK (e.g., NTRK1, NTRK2, and NTRK3), TRK proteins (e.g., TRKA, TRKB, TRKC), biomarker compounds identified in Table 1, and the like.

In some embodiments, biomarker compounds are associated with one or more diseases or disorders. For example, the identification of individual biomarker compounds in a fluid sample are used to diagnose a subject as having a disease or disorder. Examples of biomarker compounds that are associated with specific diseases or disorders are identified in Table 1.

TABLE 1

| Cancer | Biomarker |
|---|---|
| Bladder cancer | EVs, HER-2, |
| Breast cancer | HER-2, ER, PR, her2/neu, ESR1, GATA3, |
| | FOXA1, XBP1, cMYB ccND1, Ki67 |
| Colorectal carcinoma | Predictive biomarkers: PCBP1, APOE, AGT, |
| | DBP, pEGFR, PSA1, LAP3, ANXA3, serpin |
| | B5, IFIT1, FASTKD2, PIP4K2B, ARID1B, |
| | SLC25A33, CALD1, CPA3, B3GALT5, |
| | CD177, RIPK1 |
| | Prognostic biomarkers: HLAB, ADAMTS2, |
| | LTBP3, NME2, JAG2, 14-3-3β protein, |
| | HSP47, ezrin, complement C4-A, fibrinogen |
| | alpha, ERF3B, angiotensinogen, fragments |
| | of alpha-fetoprotein, collagen type XII, |
| | collagen VI, forkhead box O3, inositol |
| | polyphosphate-4-phosphatase, LcK tyrosine |
| | kinase, phosphor-PEA15 (Ser116), |
| | phosphor-PRAS40, Rad51, Phospho-S6 |
| | (ser240-244), maspin |
| | KRAS, BRAF, POLE mutations, MASPIN, |
| | SATB1, HDACs (SIRT1, HDAC2, |
| | H4K16Ac), RSPO fusions, phospholipase |
| | (PLA2G2A), exosomes (ALG-2 interacting |
| | protein X) |
| Renal cancer | NS, CK7, AMACR, Vimentin, FH, 2SC, |
| | CD117, TFE3, Cathepsin K, EMA, INI1, |
| | 34betaE12, SDHB, carbonic anhydrase (CA) |
| | IX |
| | HGF, MIG, IL-18BP, IL-18, ANG-2; TIMP- |
| | 1, M-CSF, IL-18BP, ANG-2, VEGF |
| Lung cancer | Mutations (EGFR, KRAS, BRAF, HER2, |
| | MET), Gene fusions (ALK, ROS1, RET, |
| | NTRK1, FGFR1/3, NRG1), amplifications |
| | (FGFR1, EGFR, MET, HER2) |
| | EGFR, ALK, ROS, KRAS, MET, NTRK1, |
| | FGFR, HER2, BRAF, PIK3CA, RET, |
| | DDR2, PTEN |
| Melanoma | MAGE-3, MUC-18, p97, tyrosinase |
| | BAP1, MGP, SPP1, CXCL14, CLCA2, |
| | S100A8, BTG1, SAP130, ARG1, KRT6B, |
| | GJA1, ID2, EIF1B, S100A9, CRABP2, |
| | KRT14, ROBO1, RBM23, TACSTD2, |
| | DSC1, SPRR1B, TRIM29, AQP3, TYRP1, |
| | PPL, LTA4H, CST6, |
| | PRAME, LINC |
| | S100A7, S100A8, S100A9, S100A12, PI3, |
| | CCL5, CD38, CXCL10, CXCL9, IRF1, |
| | LCP2, PTPRC, SELL, PRAME, CLTC, |
| | MRFAPI, PPP2CA, PSMA1, RPL13A, |
| | RPL8, RPS29, SLC25A3, TXNLI |
| Oral cancer | Body-fluid biomarker: AMDL DR-70, |
| | SCCA-1, CA125, CA19-9, TPS, CEA, SCC, |
| | and Cyfra 21-1, Adenosine deaminase, |
| | Adiponectin, Syndecan-1, Prolactin and TPS, |
| | EGF, IGF-1, IL-1a, IL-6, IL-8, VEGF-a, |
| | TNF-a, COL5A1, ABCG1, MMP1, FN1 |
| | Protein biomarker: Bmi-1, c-myc, Snail, |
| | Foxp3, RCAS1, Metallothionei, HDAC-1 |
| | and -2, TRB3 and p-AKT, MMP-2, MMP-8, |
| | MMP-9, and MMP-13, GOLPH3, FAK and |
| | Src, TLR5, AEG-1, EZH2 and Ki-67, |
| | BATF2, FLOT1, Eph-A1, -A2, -A4, and -A7, |
| | LAT1, ASCT2, xCT, 4F2hc, and Ki-67, |
| | α -SMA, N-cadherin, vimentin, and |
| | LYVE-1, p16, t-ERK1 and p-ERK1/2, |
| | PKM2 and LDH5, LSD1 and Ki-67, ZEB1 |
| | and CA9, CAFs and Activin A, MMP2 and |
| | MMP9, CAF, Foxc2, RKIP, MMP13 and |
| | TLR9, VEGF-C and VEGF-A, VEGF-C, |
| | VEGFR-3, and podoplanin, CB1R and |
| | CB2R, VEGF-C, VEGFR-3, CCR7, Nrp1, 2, |
| | MVD, LVD, and SEMA3E, Securin, |
| | HMGA2, Snail, E-cadherin, and Vimentin, |
| | HK2, SUZ12, pEGFR, HA and EGFR, Nrp2, |
| | VEGF-C, VEGFR-3, and Sema3F, SIP1 and |
| | E-cadherin, Snai1, Snai2, E-cadherin, and |
| | vimentin, CXCR4, CXCR12, CA9, E- |
| | cadherin, and vimentin, Snail, Twist, E- |
| | cadherin, and Ncadherin, and vimentin, HIF- |
| | 1α, HIF-2α TWIST2, and SNIP1, CypA, |
| | CD147, HIF-1 α, VEGF-A, and VEGF-C, |
| | HIF-1 α, CA-9, GLUT-1, and EPOR, HIF-1α |
| | and VEGF, SOX2, ALDH1, CD44, OCT4, |
| | and SOX2 |
| | RNA biomarker: lncRNA UCA1, lnc- |
| | AL355149.1-1, lnc-PPP2R4-5, lnc-SPRR2D- |
| | 1, lnc-MBL2-4:3, lnc-MAN1A2-1, lnc- |
| | FAM46A-1, lnc-STXBP5-1, and lnc-MBL2- |
| | 4:1, lncRNA MEG3, lncRNA HOTTIP, |
| | lncRNA NKILA, lncRNA TUG1, lncRNA |
| | TUC338, lnc RNA 152 (LINC00152), |
| | lncRNA 673 (LINC00673), MALAT1 |
| | DNA biomarker: TP53, CCND1, 7q21, |
| | MMP-1 -1607 1G/2G and IL-8 -251 A/T, |
| | Her-2/neu, EGFR, FADD, Telomeres, |
| | WIF1 and RUNX3 methylation, FGFR1, |
| | Survivin gene, STK11, MET, PIK3CA, |
| | BRAF and NRF2, CDKN2A, 8q11.21, |
| | 8q12.2-3, and 8q21.3, 22q11.23, 16p11.2, |
| | and 20q11.2, ACTN4, FHIT, EGFR, LOH, |
| | TP53 DNA binding domain, NOTCH1 |
| Pancreatic cancer | CA 19.9, CEA, ICAM-1, OPG, MIC-1, |
| | TIMP-1, S100P, KRAS, TP53, SMAD4, |
| | CDKN2A, KDM6A, PREX2, p16, ppENK, |
| | cyclin D2, SPARC/osteonectin SOCS-1, |
| | TSLC1, GNAS, BNIP3, PTCHD2, SOX17, |
| | NXPH1, EBF3, ADCY1, CD1D, BMP3, |
| | miR-1290, miR-145, miR-150, miR-223, |
| | miR-636, miR-26b, miR-34a, miR-122, |
| | miR-126, miR-145, miR-150, miR-505, |
| | miR-636, miR-885.5p, miR-138, miR-195, |
| | miR-204, miR-216a, miR-217, miR-218, |
| | miR-802, miR-155, miR-214, miR-26a, |
| | miR-30b, miR-31, miR-125, gelsolin, |
| | lumican, galectin-1, Laminin |
| Endometrial cancer | PTEN, KRAS, CTNNB, TP52, Ki-67, p16, |
| | CDH1, HER2, ARID1A, ARID5B, RPL22, |
| | PPPR1A, PIK3R1, PIK3CA, FBXW7, |
| | CTCF, POLE, CTNNB1 |
| Autoimmune Diseases or | |
| Disorders | |
| Rheumatoid arthritis | Rheumatoid factor (RF), anti-Sa, anti-CCP, |
| | anti-CarP, anti-BiP, anti-GPI, anti-MCV, 14- |
| | 3-3eta, COMP, calprotectin, survivin, ADA, |
| | RTX, TCZ, anti-TNF, IFX |
| Lupus | miR-193a-5p, miR-423, miR-501-3p, miR- |
| | 874, miR-21, miR-150, miR-29 |
| Multiple Sclerosis | Oligoclonal bands (OCB), IgG index |
| | (albumin), measles, rubella, varicella-zoster |
| | (MRZ), immunological changes related to B |
| | cell activation, anti-aquaporin-4 antibodies, |
| | anti-MOG, IFN-β, MxA, neutralizing |
| | antibodies against natalizumab, C-X-C motif |
| | chemokine-13 (CXCL13) |
| Inflammatory bowel | Serum biomarkers: ASCA, pANCA, CRP |
| disease (Crohn's | Fecal biomarkers: calprotectin, lactoferrin |
| disease and | Inflammatory biomarkers: serum amyloid A |
| ulcerative colitis) | (SAA), eotaxin-1, IL-6, IL-8, IL-17A, TNF- |
| | α |
| Neurodegenerative | |
| Disorder | |
| Alzheimer's Disease | ptau181 |
| | proteins associated with neocortical Aβ |
| | deposition |

In certain embodiments, the portable devices analyze a single sample for more than one biomarker compound. It will be generally understood that any combination of compounds may be analyzed in a sample using the portable devices described herein.

A sample may be any fluid sample received from a subject, such as saliva, blood, urine, or other bodily fluid. In some embodiments, a sample is obtained from the upper or lower respiratory tract of a subject. For example, a sample may be obtained from a nasopharyngeal/oropharyngeal swab, nasal aspirate, nasal wash, saliva, sputum or tracheal aspirate, or bronchoalveolar lavage (BAL) from a subject.

One skilled in the art will recognize that various embodiments of the disclosure discuss the analysis of nucleic acid compounds, e.g., RNA or DNA, though certain embodiments of the disclosure can also be used for the analysis of antibodies, antigens, proteins, e.g., fusion proteins, or genes.

In some embodiments, the methods described herein for identifying one or more biomarkers in a sample may include utilizing a CRISPR/Cas system. As is generally known to those of skill in the art, CRISPR/Cas systems can be divided into two main classes. Class 1 CRISPR/Cas systems utilize different types of proteins or enzymes, each having a specific role, to achieve foreign DNA cleaving. Class 2 CRISPR/Cas systems utilize a Cas protein and gRNA for DNA cleavage. The gRNA may consist of 2 RNA transcripts, crRNA and tracrRNA. crRNA defines the specificity and selectivity of the target sequence whereas, tracrRNA (trans activating RNA) guides crRNA and Cas protein to the target site. Cas may be a multi-domain protein which when complexed with gRNA forms ribonucleoprotein (RNP) surveillance complex. Class 2 systems are generally the most widely used system in bioengineering and diagnostics and can be further divided into three types namely, Type II, Type V and Type VI.

Microbial Clustered Regularly Interspaced Short Palindromic Repeats (CRISPR) and CRISPR-associated (CRISPR-Cas) adaptive immune systems contain programmable endonucleases, e.g., Cas. RNA-guided ribonucleases can be reprogrammed using CRISPR RNA (crRNAs) to cleave one or more target nucleic acids. The Cas enzyme may remain active after cleaving the RNA target, leading to “collateral” cleavage of non-targeted nucleic acids in the proximity. The sgRNA-programmed collateral nucleic acid cleavage activity presents an opportunity to use RNA-guided ribonucleases to detect the presence of a specific nucleic acid (e.g., RNA) by triggering in vivo programmed cell death or in vitro nonspecific RNA degradation that can serve as a signal identifiable by a device.

In some embodiments described herein, sgRNA targeting effectors provide a robust CRISPR-based diagnostic with attomolar sensitivity. In some aspects, biomarkers (e.g., nucleic acids) having comparable levels of sensitivity are detectable and, in some aspects, target biomarkers (e.g., nucleic acids) may be distinguishable from non-target biomarkers (e.g., nucleic acids) based on single base pair differences.

In some embodiments, the CRISPR/Cas system comprises a Cas protein or enzyme, such as Cas9, Cas12, Cas13, or any other Cas protein known to those of skill in the art. In one embodiment, the CRISPR/Cas system comprises a Type VI RNA-targeting Cas enzyme. The Type VI RNA-targeting Cas enzyme may be Cas13a or Cas13b. In some aspects, a homologue or orthologue of a Type VI protein, such as Cas13a, has a sequence homology or identity of at least 30%, at least 40%, at least 50%, at least 60%, at least 70%, at least 80%, at least 85%, at least 90%, or at least 95% with a Type VI protein such as Cas13a (e.g., based on the wild-type sequence of any one of Leptotrichia shahii Cas13a, Lachnospiraceae bacterium MA2020 Cas13a, Lachnospiraceae bacterium NK4A179 Cas13a, Clostridium aminophilum (DSM 10710) Cas13a, Carnobacterium gallinarum (DSM 4847) Cas13a, Paludibacter propionicigenes (WB4) Cas13a, Listeria weihenstephanensis (FSL R9-03 17) Cas13a, Listeriaceae bacterium (FSL M6-0635) Cas13a, Listeria newyorkensis (FSL M6-0635) Cas13a, Leptotrichia wadei (F0279) Cas13a, Rhodobacter capsulatus (SB 1003) Cas13a, Rhodobacter capsulatus (R121) Cas13a, Rhodobacter capsulatus (DE442) Cas13a, Leptotrichia wadei (Lw2) Cas13a, or Listeria seeligeri Cas13a). In other aspects, the homologue or orthologue of a Type VI protein, such as Cas13a, has a sequence identity of at least 30%, at least 40%, at least 50%, at least 60%, at least 70%, at least 80%, at least 85%, at least 90%, or at least 95% with the wild type Cas13a (e.g., based on the wild-type sequence of any one of Leptotrichia shahii Cas13a, Lachnospiraceae bacterium MA2020 Cas13a, Lachnospiraceae bacterium NK4A179 Cas13a, Clostridium aminophilum (DSM 10710) Cas13, Carnobacterium gallinarum (DSM 4847) Cas13, Paludibacter propionicigenes (WB4) Cas13a, Listeria weihenstephanensis (FSL R9-03 17) Cas13a, Listeriaceae bacterium (FSL M6-0635) Cas13a, Listeria newyorkensis (FSL M6-0635) Cas13a, Leptotrichia wadei (F0279) Cas13a, Rhodobacter capsulatus (SB 1003) Cas13, Rhodobacter capsulatus (R121) Cas13a, Rhodobacter capsulatus (DE442) Cas13a, Leptotrichia wadei (Lw2) Cas13a, or Listeria seeligeri Cas13a).

In some embodiment, the Cas protein may be a Cas13a orthologue of an organism of a genus which includes but is not limited to Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter.

In some embodiments, the Cas13a protein may be from an organism selected from the group consisting of Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, and Campylobacter.

The Cas protein may also encompass a functional variant of the Cas protein or a homologue or an orthologue thereof. A “functional variant” of a protein as used herein refers to a variant of such protein which retains at least partial activity of that protein.

In one embodiment, nucleic acid molecule(s) encoding the Cas protein or an orthologue or homolog thereof, may be codon-optimized for expression in a eukaryotic cell.

In one embodiment, the Cas protein or an orthologue or homolog thereof, may comprise one or more mutations. The mutations may be artificially introduced mutations and may include, but are not limited to, one or more mutations in a catalytic domain.

In one embodiment, the Cas protein or an orthologue or homolog thereof, may be used as a generic nucleic acid binding protein with fusion to or being operably linked to a functional domain. Exemplary functional domains may include, but are not limited to, translational initiator, translational activator, translational repressor, nucleases, ribonucleases, a spliceosome, beads, a light inducible/controllable domain or a chemically inducible/controllable domain.

In some embodiments, the CRISPR/Cas system comprises one or more guide RNA (gRNA or sgRNA). Guide RNA may be a combination of trans-activating CRISPR RNA (tracrRNA) and a CRISPR RNA (crRNA) designed to cleave the gene target site of interest or may be a single guide RNA (sgRNA) that consists of both the crRNA and tracrRNA as a single construct. As used herein, the term “crRNA,” “gRNA,” “guide RNA,” “single guide RNA,” or “sgRNA” refers to a polynucleotide comprising any polynucleotide sequence having sufficient complementarity with a target nucleic acid sequence to hybridize with the target nucleic acid sequence and to direct sequence-specific binding of a RNA-targeting complex comprising the gRNA and a CRISPR effector protein (e.g., Cas) to the target nucleic acid sequence.

In some embodiments, the CRISPR/Cas system comprises crRNA or an analogous polynucleotide comprising a guide sequence, wherein the polynucleotide is an RNA, a DNA or a mixture of RNA and DNA, and/or wherein the polynucleotide comprises one or more nucleotide analogs. In some aspects, the guide sequence has any structure, including but not limited to a structure of a native crRNA, such as a bulge, a hairpin or a stem loop structure (e.g., a single stem loop). In one aspect, a direct repeat sequence forms a stem loop (e.g., a single stem loop). In one embodiment, the polynucleotide comprising the guide sequence forms a duplex with a second polynucleotide sequence which can be an RNA or a DNA sequence.

A guide sequence, and hence a nucleic acid-targeting guide RNA may be selected to target any target nucleic acid sequence. In the context of formation of a CRISPR complex, “target sequence” refers to a sequence to which a guide sequence is designed to have complementarity, where hybridization between a target sequence and a guide sequence promotes the formation of a CRISPR complex.

In some embodiments, a biomarker detection system (e.g., a nucleic acid detection system) comprises one or more elements derived from a particular organism comprising an endogenous CRISPR/Cas system. In some aspects, the effector protein of the CRISPR/Cas system comprises at least one HEPN domain, including but not limited to HEPN domains known in the art and domains recognized to be HEPN domains by comparison to consensus sequence motifs.

In some embodiments, a biomarker detection system (e.g., nucleic acid detection system) comprises a CRISPR/Cas system comprising an effector protein (e.g., a RNA targeting effector protein such as Cas) and one or more guide RNAs where the gRNA is designed to bind to a corresponding target sequence. In some embodiments, the detection system further comprises one or more detection aptamers. The one or more detection aptamers may comprise an RNA polymerase site or a trimer binding site. In some aspects, the one or more detection aptamers bind one or more target polypeptides and are configured such that the RNA polymerase site or trimer binding site are masked and are exposed only upon binding of the detection aptamer to the target polypeptide. In some aspects, exposure of the RNA polymerase site facilitates generation of a trigger RNA oligonucleotide using the aptamer sequence as a template. Accordingly, in such embodiments, the one or more guide RNAs are configured to bind to a trigger RNA. In some aspects, the one or more gRNAs bind to a target sequence, e.g., a target nucleic acid, which in turn activates the CRISPR effector protein, e.g., Cas. Once activated, the CRISPR effector protein then deactivates the masking construct, for example, by cleaving the masking construct such that a detectable positive signal is unmasked, released, or generated. Detection of the positive detectable signal may indicate the presence of the target molecules.

In some embodiments, a biomarker detection system (e.g., a polypeptide detection system) comprises a CRISPR/Cas system comprising an effector protein (e.g., a RNA targeting effector protein such as Cas) and one or more guide RNAs where the gRNA is designed to bind to a corresponding target sequence. In some embodiments, the detection system further comprises one or more peptide detection aptamers. The one or more peptide detection aptamers may facilitate the generation of a trigger oligonucleotide upon binding to a target polypeptide. Accordingly, in such embodiments, the one or more guide RNAs are configured or designed to recognize the trigger oligonucleotides, thereby activating the CRISPR effector protein. In some aspects, deactivation of the masking construct by the activated CRISPR effector protein leads to unmasking, release, or generation of a detectable positive signal.

In some aspects, the CRISPR/Cas system or the guide RNA reagents are preloaded into individual discrete volumes or are added to individual discrete volumes concurrently with or subsequently to the addition of a sample to each individual discrete volume. In some aspects, transgenic cells may comprise the CRISPR/Cas system. The transgenic cell may function as an individual discrete volume. For example, samples comprising a masking construct may be delivered to a cell, such as in a suitable delivery vehicle, and if the target is present in the delivery vehicle the CRISPR effector is activated and a detectable signal is generated. In some aspects, a device, e.g., an electrochemical biosensor-based device, may define the individual discrete volumes.

Disclosed herein are portable devices and methods for detecting and identifying one or more compounds in one or more sample specimens of a subject. Also disclosed herein are immobilization methods with different antibodies for biosensor substrates. In some embodiments, the CRISPR/Cas system described herein is utilized in the detection and identification of one or more compounds in one or more sample specimens of a subject.

In one embodiment, the CRISPR/Cas system is used to detect RNAse activity electrochemically via cleavage of RNA-tethered inhibitors. Many common enzymes are known to have competitive, reversible inhibitors, which may be weak. However, the effect of the inhibitors can be increased by increasing the local concentration. By linking the local concentration of inhibitors to RNAse activity, electrochemical enzyme and inhibitor pairs can be engineered into RNAse sensors. The electrochemical RNAse sensor based upon small molecule inhibitors involves three components: the electrochemical enzyme, the inhibitor, and a bridging RNA that is covalently linked to both the inhibitor and enzyme, thereby tethering the inhibitor to the enzyme. In the uncleaved configuration, the enzyme is inhibited by the increased local concentration of the small molecule inhibitor; however, when the RNA is cleaved (e.g. by Cas13a collateral cleavage), the inhibitor is released, and the electrochemical enzyme is activated.

In one embodiment, a masking construct binds to an immobilized reagent in solution thereby blocking the ability of the reagent to bind to a separate labelled binding partner, e.g., that is free in solution. In some aspects, upon application of a washing step to a sample, the labelled binding partner is washed out of the sample in the absence of a target molecule. However, if an effector protein is activated, the masking construct is cleaved to a degree sufficient to interfere with the ability of the masking construct to bind the reagent thereby allowing the labelled binding partner to bind to the immobilized reagent. Thus, the labelled binding partner remains after the wash step thereby indicating the presence of the target molecule in the sample. In certain aspects, the masking construct that binds the immobilized reagent is an RNA aptamer.

In some aspects, the immobilized reagent is a protein and the labelled binding partner is a labelled antibody. In alternative aspects, the immobilized reagent is a streptavidin and the labelled binding partner is labelled biotin. In some aspects, the label on the labelled binding partner is any detectable label known in the art. In addition, other known binding partners may be used in accordance with the overall design described here.

In some embodiments, a target biomarker, e.g., target nucleic acid, may be enriched prior to electrochemical detection of the target nucleic acid (e.g., RNA or DNA). In some embodiments, enrichment is achieved by the binding of the target nucleic acids by a CRISPR system.

In some embodiments, a dead CRISPR protein that Cas may bind the target nucleic acid in solution and then subsequently be isolated from said solution. For example, the dead CRISPR protein bound to the target nucleic acid, may be isolated from the solution using an antibody or other molecule, such as an aptamer, that specifically binds the dead CRISPR protein.

In one embodiment, target nucleic acids may be released from the CRISPR/Cas system for further detection using the methods disclosed herein. In some aspects, the target nucleic acids are detected electrochemically as described herein, e.g., using the portable devices and methods described herein. In some aspects, the target nucleic acid comprises one or more mutations. In one aspect, one or more mutations may comprise two mutations. In a preferred embodiment the one or more amino acid residues are modified in a Cas13a effector protein, e.g., an engineered or non-naturally occurring effector protein or Cas13a.

In some embodiments, the methods for identifying one or more biomarkers (e.g., nucleic acids) in a sample, as described herein, comprise distributing one or more samples into individual discrete volumes, each individual discrete volume comprising peptide detection aptamers, a CRISPR effector protein (e.g., Cas), and one or more guide RNAs. The one or more samples may then be incubated under conditions sufficient to allow binding of the peptide detection aptamers to the one or more target molecules, wherein binding of the aptamer to a corresponding target will expose the RNA polymerase promoter binding site resulting in synthesis of a trigger RNA via binding of a RNA polymerase to the RNA polymerase promoter binding site.

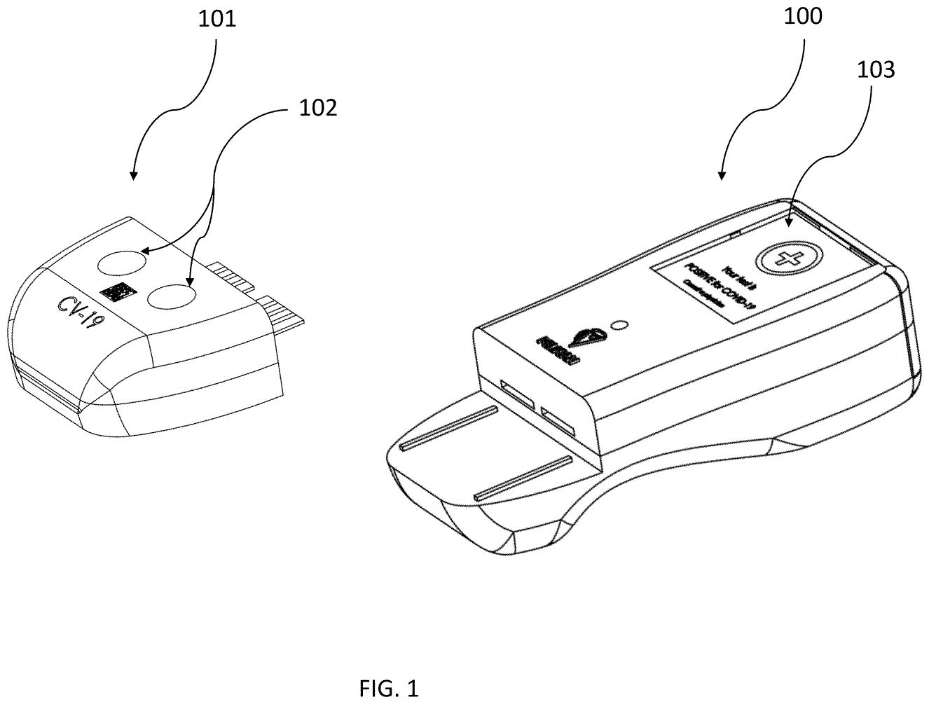

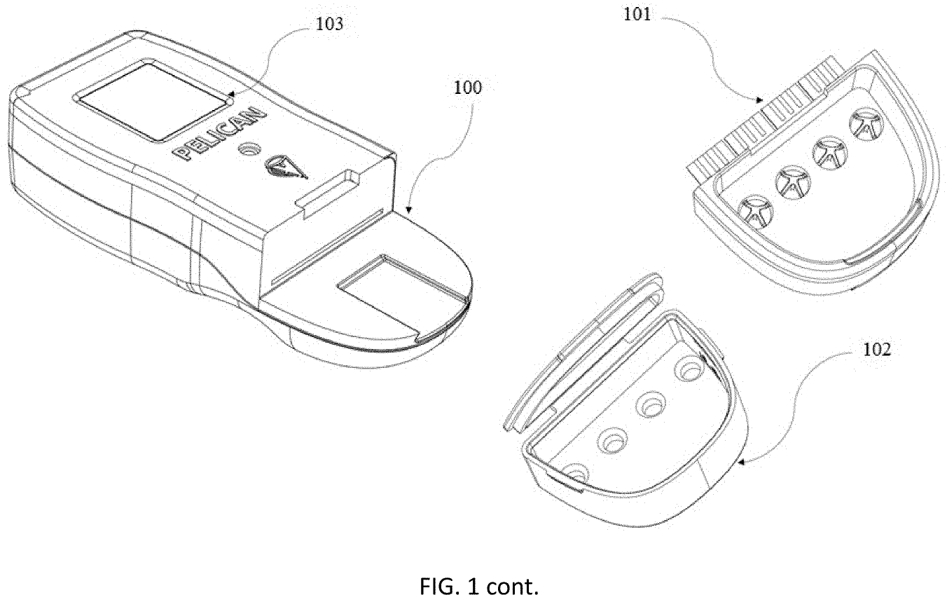

An example of a portable device is shown in FIG. 1. The portable device may be used to detect and identify one or more compounds in a sample specimen from a subject. A portable device 100 may be used to detect and identify one or more nucleic acids in a sample from a subject. Portable device 100 contains sensor cartridge 101 which includes a sample well 102, and a hub station with display 103. The sample well may include a spit-cup. Other designs and configurations of such a portable device are possible and the above illustration is not limiting. For example, the sensor cartridge may comprise one or more biosensors. In some aspects, each biosensor may identify one or more targets, for example, one or more variants of a virus or one or more viruses may be detected on a single biosensor. In other aspects, a portable device may comprise one or more biosensors and each biosensor may be used to detect one or more variants of a virus or one or more viruses.

Briefly, a portable device, such as the one illustrated in FIG. 1, may collect data about the presence and identity of one or more biomarker compounds, such as nucleic acids, in a sample from a subject with a sensor module. The biomarker compounds may be associated with a disease or disorder (e.g., a virus or a cancer). The sensor module may convert the collected data to a signal (e.g., provides significantly high electrical conductivity, thermal conductivity, optical etc.), and transmits the data via a communication apparatus to a processing apparatus which analyzes the data to provide information about the presence and identity of the biomarker compounds in the sample from the subject and, in some aspects, may transmit the presence and identity of the one or more biomarker compounds to a display on the portable device.

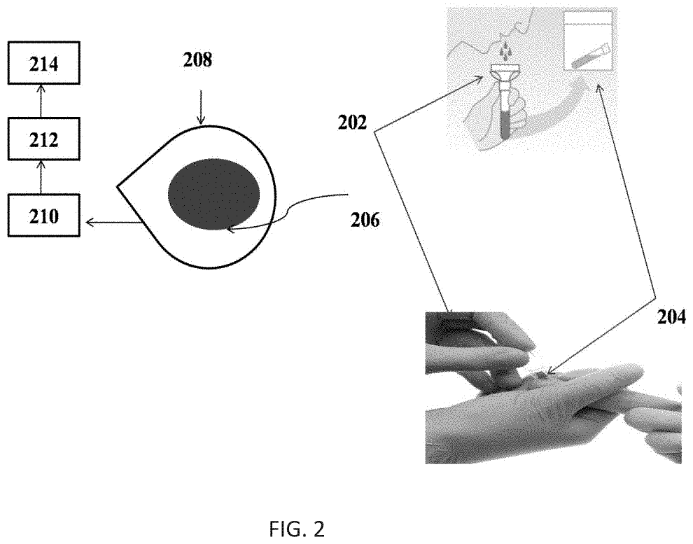

The process described above is illustrated in detail in FIG. 2. Subject 202 deposits, for example, a sample specimen 204 including one or more nucleic acids into housing 208, via sample well 206. In some aspects, the sample specimen is breath, saliva, blood, urine, or other bodily fluid. In some aspects, the sample enters the housing 208, which includes a battery, a sensor module, and a communication apparatus, which transmits the data (e.g., by wireless or electrical means) collected by the sensor module, for example, to the cloud 210. The data may be stored in the cloud 210 and may be accessed by processing apparatus 212, which detects and identifies the compound in the sample of the subject and communicates the result via a display to a user 214. The communication apparatus may directly transmit data to the processing apparatus 212, thus bypassing cloud 210. In certain embodiments, the process illustrated in FIG. 2 takes place in real time (i.e., at substantially at the same time the event is occurring, with any delay being less than, for example, one minute). In some aspects, data may be stored in the cloud before being processed by the processing apparatus and displayed to a user 214.

The sample of a subject is inserted into an inlet or a sample well to enter the sensor module. In certain embodiments, sensor module will be positioned in the housing to receive the sample specimen of the subject. The sensor module may include biosensors, which are independently capable of detecting the presence one or more biomarkers, e.g., genes and nucleic acids, in the sample of the subject. In some embodiments, the biosensors are attached to a base, which is, in certain embodiments, molded plastic or ceramic. In some embodiments, the sensors are selected to detect the presence of one or more biomarkers, e.g., genes and nucleic acids, in the sample of the subject. In one embodiment, sensor modules are replaceable, which allows the portable device to be rapidly adapted to recognize the presence and identity of specific types of biomarkers, e.g., genes, nucleic acids, and proteins.

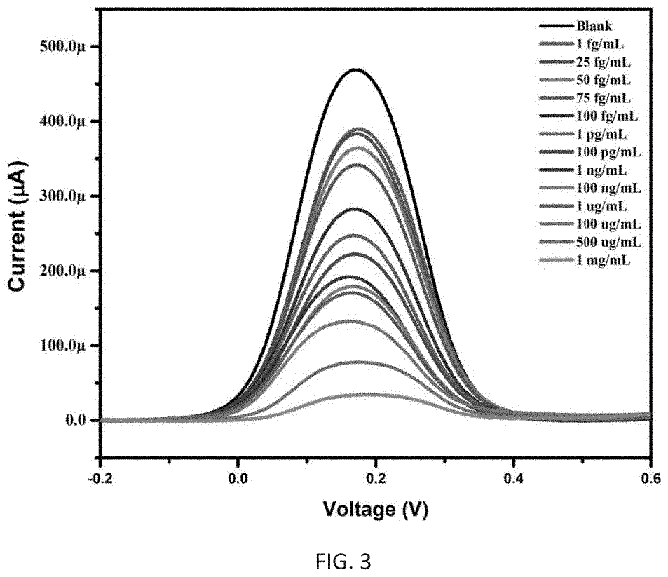

Those of skill in the art will appreciate that compositions of immobilized protein with nucleic acid marker compounds at varying concentrations can be used to create a vast library of sequential infection and disease detection. For example, protein which includes the immobilized screen-printed gold electrode can be detected at different concentrations ranging from 1 fg to 1 μg can be readily distinguished as illustrated in FIG. 3.

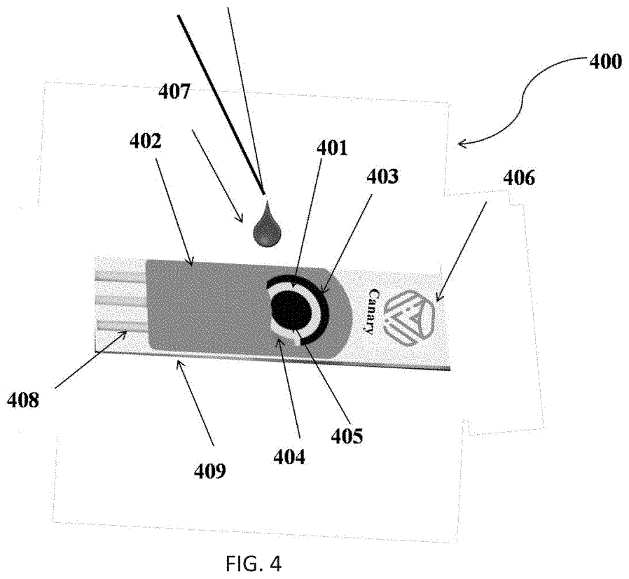

A microfluidic, electrochemical biosensor as described in certain embodiments is shown in FIG. 4. In one embodiment, a portable device includes a biosensor, also referred to herein as an electrochemical subsystem 400, including amplification and detection reagents for electrochemical detection. In certain aspects, the biosensor is a biosensor cartridge that is insertable into an electrochemical reader for electronic readings. In one aspect, the biosensor cartridge is disposable. In one aspect, the biosensor cartridge is reusable. In some aspects, the electrochemical reader may detect one or more signals selected from the group consisting of electrical current, voltage, and other electric signals known in the art. As exemplified in FIG. 4, electrochemical subsystem 400 includes a first cellulosic layer 402 which includes one or more amplification agents in a sample deposition zone 401. The use of cellulosic layer (e.g., paper)-based test strips reduces the sample volume and therefore the cost of reagents. In some embodiments, the cellulosic layer is a patterned layer, e.g., a patterned paper layer. For example, the sample deposition zone 401 is a hydrophilic porous area in the cellulosic layer defined by a fluid-impermeable material which permeates through the thickness of first cellulosic layer 402 and surrounds the sample deposition zone 401. Electrode assembly 408 may contain a three electrodes system that is counter electrode 403, reference electrode 404 and working electrode 405. In some aspects, electrode assembly 408 comprises 1 to 10 working electrodes 405, or in some aspects 4 to 8 working electrodes 405. In one embodiment, electrode assembly 408 includes at least one reference electrode 404 and at least one counter electrode 403. In one embodiment, the individual electrodes in electrode assembly 408 are, for example, screen-printed electrodes (SPE) 406. Screen-printed electrodes 406 connect electrically to a set of contact pads 409 from the test zone 407. The sample deposition zone 401 is in fluidic communication with test zone 407 on first cellulosic layer 402. In certain embodiments, one or more amplification agents are embedded in the test zone 407 and/or the sample deposition zone 401. Amplification agents interact with the genetic material to provide copies of the genetic material using one of many methods disclosed herein. Non-limiting examples of amplification agents include T4 RNA ligase, DNA polymerase, RNA polymerase, t4 pnk, etc. In certain embodiments, one or more binding agents are embedded in the test zone 407 and/or the sample deposition zone 401. The binding agents may be selected for their ability to bind the amplified genetic material to result in a change of the concentration of signaling chemicals. Non-limiting examples of binding includes include carbodiimide compounds (EDC, MHS), carboxyl-reactive crosslinker reactive groups, etc. The test zone 407 is in fluidic communication with the electrode assembly 408. In some embodiments, a hydrophilic channel connects the test zone to the electrode assembly fluidically. In other embodiments, at least part of the electrode assembly is located in, printed in, or overlaps with the test zone. When the signaling chemical's concentration is changed as a result of the binding between the binding agents and the amplified genetic material, such a change may be detected by the electrochemical reader to generate an electronic readout. Other variations in the arrangement of the cellulosic layers, sample deposition zone and detection zone are contemplated and will be apparent to one of skill in the art.

In one embodiment, an electrochemical biosensor identifies and detects one or more biomarker compounds in a sample specimen. In some embodiments, the compound is an antibody, protein (e.g., fusion protein), nucleic acid (e.g., DNA or RNA), or gene in the saliva or blood of a subject. In some embodiments, the biomarker compound is a nucleic acid. In some embodiments, the biomarker compound is a fusion gene. In some embodiments, the biomarker compound is a viral nucleic acid. In one embodiment, the biomarker compound is a guide RNA (gRNA) for spike (S) and nucleocapsid (N) protein and/or muted genes (N-gene) for wild type or variants organisms. In another embodiment, the biomarker compound is NTRK. In another embodiments, the biomarker compound is ptau protein.

In some aspects, a sample encounters an electrochemical sensor which may include a counter electrode, a plurality of working electrodes (e.g., two or four working electrodes), and a reference electrode. The counter electrode is, for example, made of carbon paint, while the reference electrode is, for example, made of silver paint. In other aspects, counter and reference electrodes may be made of boron doped diamond, silver (Ag) or platinum (Pt). Electrodes which may be used in the sensors described herein include, but are not limited to, multiwalled carbon nanotubes (MWCNT)/Ag nanohybrids/Au, Ag nanoparticles/DNA/glassy carbon electrodes (GCE), nano-CuO/Ni/Pt, PtPdFe₃O₄ nanoparticles/GCE, CO₃O₄ nanoparticles/GCE, Cu2O/Ni/Au, AgMnO₂-MWCNTs/GCE, immobilized with antibody with carbon nanostructure or graphene gold nanocomposites, etc. In some aspects, application of voltage to working electrodes, which includes carbon paint and multi-wall carbon nanotubes, leads to recognition of the presence and identity of one or more biomarker compounds, e.g., nucleic acids, in the sample specimen of the subject.

Gold nanoparticles (AuNPs) are the most stable metal nanoparticles, due to their unique optical, electrical, and catalytic activity, as well as high biocompatibility properties and enhanced electron transfer rate. Therefore, they have shown wide applications in various electrochemical biosensors. Gold nanoparticles can be prepared by the chemical or electrochemical reduction of gold salt. Electrode deposition of AuNPs on the surface of carbon electrodes is appealing due to its direct, fast and easier preparation method.

Other materials which may be used in working electrodes include but are not limited to immobilized antibody gold nanoparticles, graphene, carbon nanotubes, reduced graphene, graphene oxide, carbon nanofibers, quantum dots, fullerene, carbon polymer nanocomposites, glassy carbon, carbon fiber nanocomposite, carbon black, etc. In an exemplary biosensor, such as that illustrated in FIG. 3, the working electrode is gold coated on a ceramic chip with an intervening insulating layer. Other supports for disposing the electrodes are generally known to those of skill in the art.

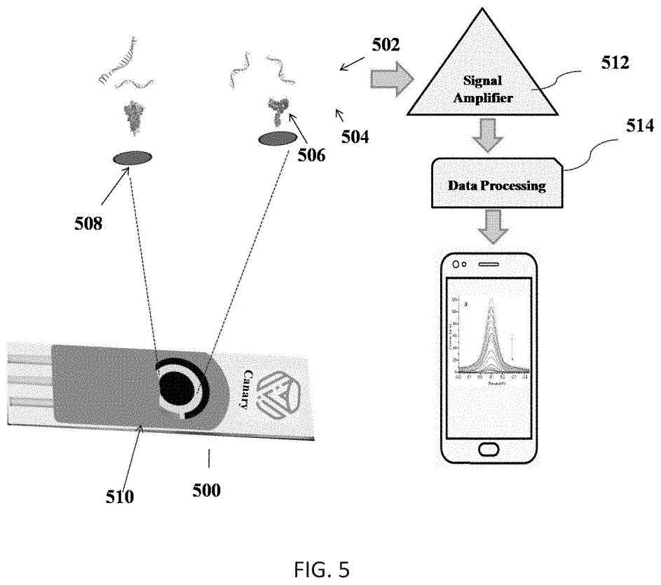

An exemplary sensor for use in a biosensor module as described herein is illustrated in FIG. 5. Sensor 500 includes one or more biological molecules 506 immobilized on support 508. Biological molecules or receptors 506 include, for example, enzymes, cells, protein, antibodies, nucleic acids, etc. Exemplified in FIG. 5 are constituents 502 which are present in a specimen sample but do not bind to immobilized biological molecules 506 on support 508. Also shown in FIG. 5 is compound 504 which specifically binds to biological molecule 506. In some embodiments, upon binding of compound 504 to immobilized biological molecule 506, a signal is generated which is communicated from transducer 510 to communication apparatus (not shown) and then to processing apparatus 514, via signal amplifier 512, where the data is processed to confirm the presence and identity of the compound. In some aspects, signal amplifier 512 reduces instability and noise and may be purchased from commercial sources. Optionally, the signal may be directly communicated to the processing apparatus without the intermediacy of a signal amplifier.

In one embodiment, a sensor or biosensor is equipped with a CRISPR/Cas system. In one embodiment, the sensor may include a Cas protein, such as Cas9, Cas12, Cas13 (e.g., Cas13a), or any other Cas protein known to those of skill in the art, and crRNA or gRNA. In some aspects, the sensor further includes a thiolated or aminated reporter sequence, e.g., a thiol functionalized reporter sequence. In one embodiment, a sensor comprising gold surfaces were functionalized with Cas13a/Cas12a protein, a thiol functionalized reporter sequence, and crRNA chemical modifications or one or more gRNA, e.g., for SARS-CoV-2 N RNA, SARS-CoV-2 S RNA, or Orflab gene region of SARS-CoV-2. In one embodiment, the sensor is analyzed using methylene blue tagged reported RNA or DNA reporter sequence which is thiol group functionalized at one terminal using square wave voltammetry to detect viral RNA (e.g., SARS-CoV-2 viral RNA) and/or mutated RNA and/or DNA sequence (e.g., those found in various types of cancer). The detection of these compounds may occur with high femtomolar detection limits with a total assay time of less than 15 minutes, and in some aspects in less than 2 minutes.

In some embodiments, a sensor, e.g., a biosensor, based on biological materials can be constructed by covalently attaching a biological molecule to a carbon nanostructure component of the working electrodes. In some embodiments, the biological molecule is an enzyme. In other embodiments, the biological molecule is an oxidase. In still other embodiments, the biological molecule is antibodies, genes, RNA, DNA, protein, or synthetic aptamers.

In some embodiments, a base material of a multilayer biosensor includes a graphene-polymer nanocomposite. The graphene-polymer composite may be coated on to the biosensor to make it suitable for absorbing the fluid component of a sample. In some embodiments, electrochemical immunosensing is based on the principle of measuring the changes in electrical properties of a conductive material due to the adsorption of an analyte on the surface functionalized with antibodies, aptamers, or nucleic acids.

In one embodiment, electrochemical impedance spectroscopy and/or cyclic voltammetry are utilized to detect one or more compounds on the sensor surface. In one embodiment, one or more electrochemical techniques are used to detect the one or more compounds, such as differential pulse voltammetry (DPV) or square wave voltammetry (SWV).

In one embodiment, DC voltage of 0.01 to 0.5V, or in some aspects 0.5 to 0.8V, is applied directly to the microcontroller embedded in the sensor module which then transfers signals to the communication device and display device. Electrical signals may monitor via a logic made in MATLAB or Python in real-time fashion. Readings record in terms of electric current (μA). In some aspects, electrical resistance can be displayed into current and voltage gain if required by reconfiguring the program for the microcontroller.

An exemplary battery is a lithium ion battery, which are conventional and available from many commercial sources (e.g., Panasonic DMW-BCM14 battery). Many batteries are known in the art and may be used in the portable devices described herein.

In some embodiments, the results from the sample analysis may be transmitted to another device, such as a conventional general-purpose computer which includes a display device and a communication interface which allows reception and transmittal of information from other devices and systems via any communication interface. Any general-purpose computer known in the art which has sufficient processing power to analyze data provided by the sensor module may be used in conjunction with the portable devices described herein. In one embodiment, the processing apparatus will detect and identify the biomarker compound in the sample specimen of the subject by processing the data received from the sensor module with the results being sent to a display device.

In some embodiments, data from biosensors in the sensor module is analyzed using pattern and recognition systems such as, for example, artificial neural networks, which include, for example, multi-layer perception, generalized regression neural network, fuzzy inference systems, etc., and statistical methods such as principal component analysis, partial least squares, multiples linear regression, etc. Artificial neural networks are data processing architectures that use interconnected nodes (i.e., neurons) to map complex input patterns with a complex output pattern. Importantly, neural networks can learn from using various input output training sets.

An exemplary artificial neural network can process data received from the sensor module. In general, the neural network can use three different layers of neurons. The first layer is input layer, which receives data from sensor module, the second layer is hidden layer while the third layer is output layer, which provides the result of the analysis. In some aspects, each neuron in the hidden layer is connected to each neuron in the input layer and each neuron in the output layer. In the exemplified neural network, hidden layer processes data received from input layer and provides the result to output layer. Although only one hidden layer is described herein, any number of hidden layers may be used, with the number of neurons limited only by processing power and memory of the general-purpose computer or smart phone. In certain aspects, the inputs to the input neurons are inputs from the sensors in the sensor module. If, for example, seven sensors are in the sensor module, then the input layer will have seven neurons. In general, the number of output neurons corresponds to the number of compounds that the sensor module is trained to detect and identify. The number of hidden neurons may vary considerably. In some embodiments, the number of hidden neurons is between about 4 to about 10.

In some embodiments, the one or more biomarker compounds (e.g., nucleic acids) which are detected and identified using the portable devices are detected and identified directly by using a specific target sequence. For example, if a specific viral RNA is ingested by a subject it may be detected and identified via gRNA for S and N protein and/or mutated genes by the portable device, i.e., a target gene may be detected using gRNA against a specific sequence. Commercial gold standard which may be directly identified and detected include, but are not limited to, is gRNA for S and N protein/muted genes, Cas12a/Cas13a and reporter sequences.

An exemplary mobile communication device can include one or more processors; a memory device including the biomarker processing module or engine, a diagnostic module, and an operating system (O/S); one or more activity sensors; a network and input/output (I/O) interface; and an output display. The example remote server computers can include one or more processors; a memory device including a biomarker processing module or engine, a diagnostic module, an operating system (O/S), and a database management system (DMBS); a network and input/output (I/O) interface; and an output display.

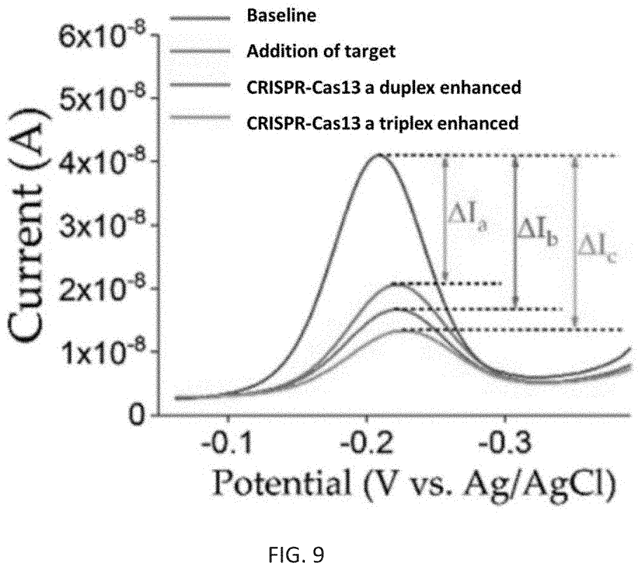

Those of skill in the art will appreciate that compositions of immobilized protein with nucleic acid marker compounds at varying concentrations can be used to create a vast library of information for use in the identification of specific biomarker compounds. In some embodiments, biomarker compounds are useful for diagnosis, monitoring disease progression, and predicting disease recurrence. For example, bioreceptors which are immobilized on screen-printed gold electrodes can be used to detect different concentrations of a biomarker (e.g., protein, RNA, DNA, aptamer, genes, etc.) ranging from lfg to lug can be readily distinguished as illustrated in FIG. 3. In some aspects, bioreceptors which are immobilized on screen-printed gold electrodes can be used to detect different concentrations of a sample/Cas/sgRNA triple complex that can be readily distinguished as illustrated in FIG. 9.

In some embodiments, the devices and methods described herein are used to diagnose a subject as suffering from or having suffered from a viral infection. In certain embodiments, the devices and methods described herein are used to diagnose or identify a patient as having or had a coronavirus. For example, a subject may be diagnosed as suffering from or recovering from a SARS-CoV-2 infection. In one embodiment, a sample specimen from the subject is positive for SARS-CoV-2 biomarkers (e.g., proteins, DNA, RNA, aptamer, genes, etc.).

In some embodiments, the biosensor cartridge comprises a first biosensor or biosensor array that includes a customized molecular probe using a specific and customized aptamer sequence for very low level and highly accurate detection of COVID-19 (e.g., via SARS-CoV-2 nucleic acids) and further comprises a second biosensor or biosensor array that includes a customized probe for the detection of protein targets. In one embodiment, multiple biomarkers are detected, i.e. protein, aptamers, gene, DNA, and/or RNA, by substituting the bioreceptor immobilized on the sensor cartridge.

In one embodiment, the devices and methods described herein are used to monitor the administration and response of a therapeutic regimen to a subject. For example, a subject receiving treatment for a disease may be monitored by having samples taken consecutively over a period of time, e.g., daily, weekly, or monthly, to monitor the viral load within the sample to determine if the subject is improving from the disease. In addition, a subject receiving treatment for a disease may be monitored by having samples taken over a period of time, e.g., daily, weekly, or monthly, to confirm the subject is maintaining a specific therapeutic regimen, e.g., samples may be taken and screened for compounds signifying specific medication is being taken by the subject. For example, a subject may be monitored to confirm the subject is maintaining a therapeutic regimen of dabigatran, rivaroxaban, apixaban, or Levicetam.

Many modifications and other embodiments of the example descriptions set forth herein to which these descriptions pertain will come to mind having the benefit of the teachings presented in the foregoing descriptions and the associated drawings. Thus, it will be appreciated that the disclosure may be embodied in many forms and should not be limited to the example embodiments described above. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### EXEMPLIFICATION

### Portable Device Example

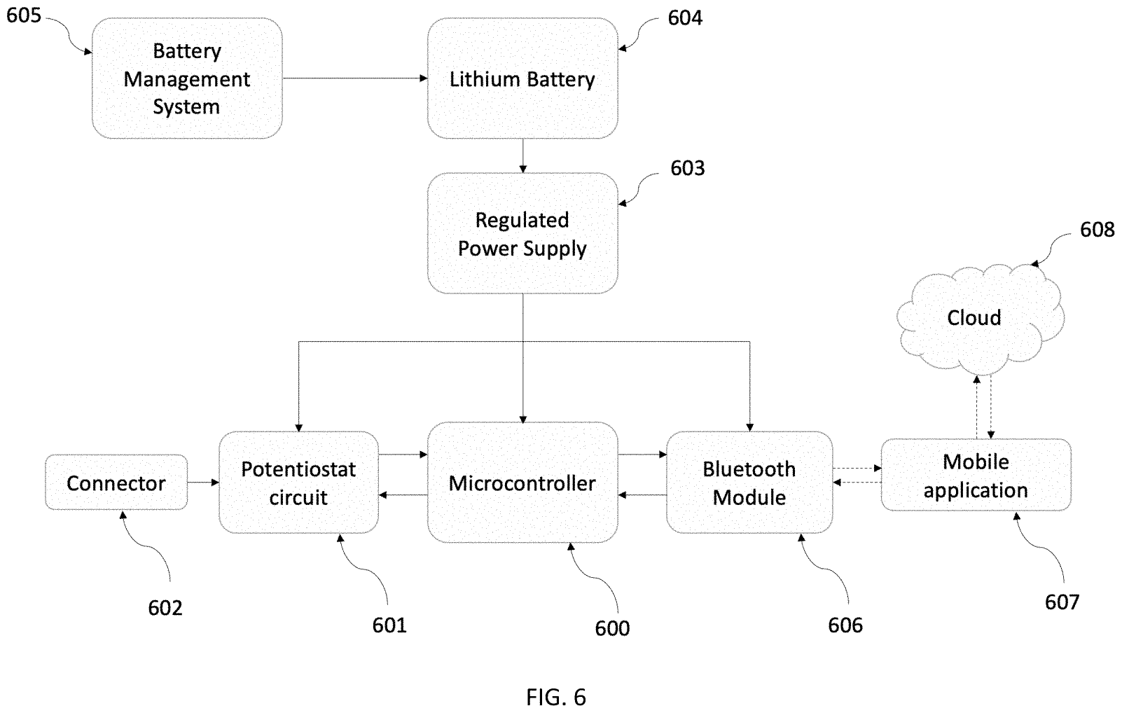

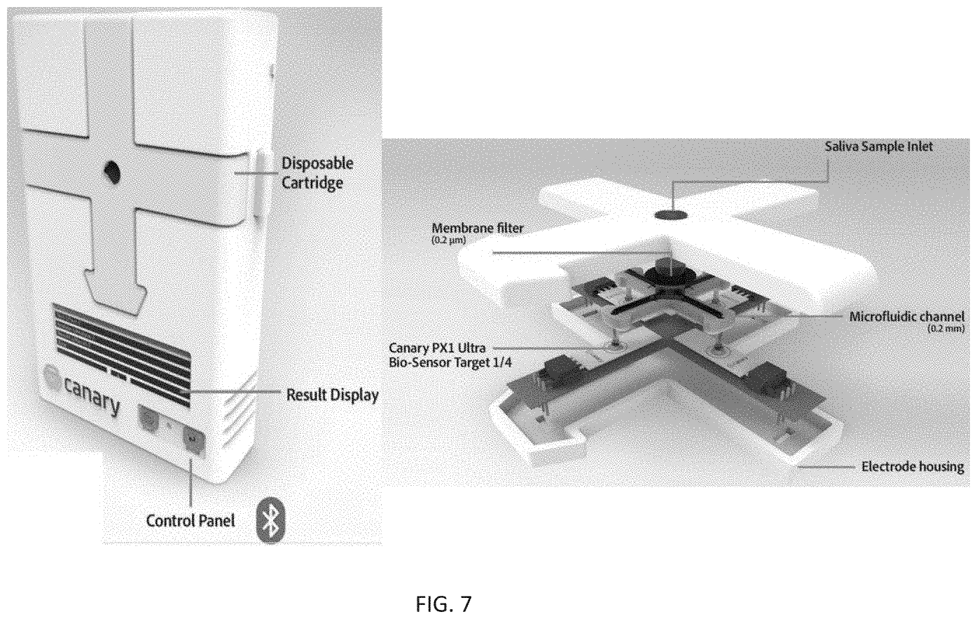

FIG. 6 provides a functional description of the different sections in the electronics which enable the measurement of electrochemical current using discrete and integrated electronic component. 601 represents the potentiostat section which subjects the electrochemical cell to a potential and records the resulting current at each potential. Potentiostat 601 provides a defined analog signal to the cell under test. This signal has a defined voltage potential and frequency. This voltage potential is applied via the counter electrode. Current response of the cell is measured on the working electrode against the reference electrode. The raw analog signal from the potentiostat signal is fed to microcontroller 600 as a part of acquisition and further processing. Microcontroller converts the analog signal into digital signal using an analog to digital convertor. This digital data is then put in a packet format and is ready for wireless transmission. A wireless feature is enabled using a Bluetooth module 606. Bluetooth module makes use of 2.4 GHz spectra for the transfer of digital data. Use of Bluetooth enables both transmission and reception of data between the device and mobile application. Bluetooth Module 606 enables the device to become truly wireless by enabling the transmission and reception of digital data between the main hardware circuitry and mobile application 607. Data from the mobile application 607 can be sent to the Cloud 608 for further data analysis. Electronic circuitry is powered using rechargeable Lithium battery 604. The use of Lithium battery 604 provides a DC (Direct Current) source of power. This source of power is passed through a Regulated Power Supply block 603 which provides the required regulated DC supply for the different peripheral blocks. Lithium battery 604 is under the control of Battery Management System 605. This ensures battery health is monitored on regular intervals. Battery management performs the function of charging, over charge-protection and short protection.

### Protocol Example

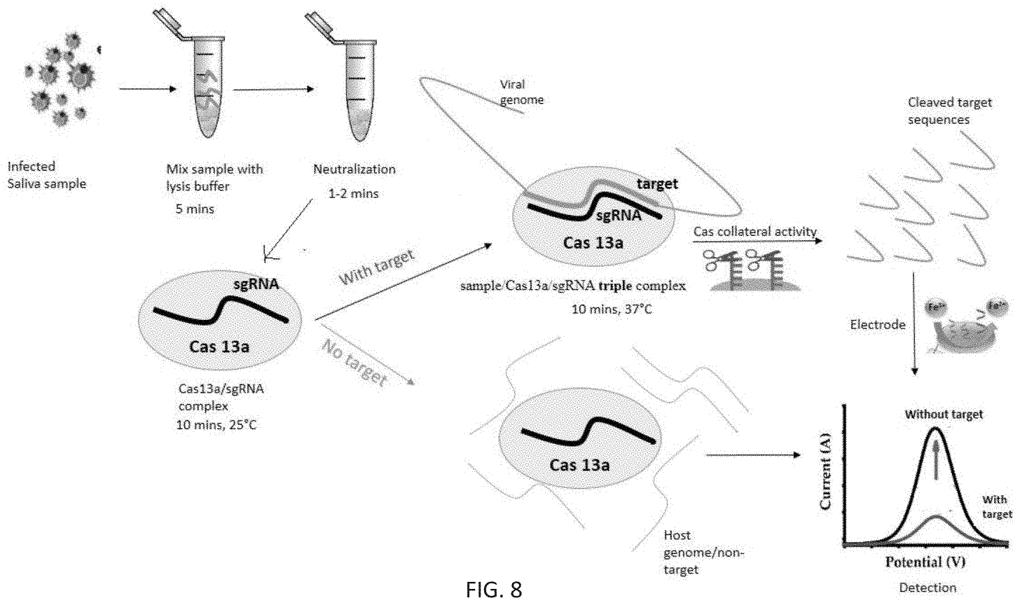

There are four steps to perform a Cas13a diagnostic test for targeted RNA. See FIG. 8 for exemplification. The protocol may also be used with protein detection variants after delivery of the detection infected RNA. In the last step where electrochemically detection and Cas13a detection are done separately.

Step 1: Obtain sample in an Eppendorf tube A (generally 1 to 3 ml) and add commercially available Lysis buffer (500 μL to 1 ml ) followed by incubation of approx. 5 mins. To neutralize the effect of lysis buffer, add PBS or Potassium acetate or other neutralizing solution in 1:1 ratio (1-2 mins).

Step 2: Prepare Cas13a/sgRNA duplex by combining in Tube B Cas13a (10-50 nM) in a Tris-NaCl buffer or other duplex promoting buffer and sgRNA (1 nM), incubate at 25° C. for approx. 5-10 mins.

Step 3: Mix the sample (tube A) with Cas13a/sgRNA (tube B) to form sample/Cas13a/sgRNA triple complex. Generally, sample and Cas13a/sgRNA duplex is mixed in a ratio of 1: 6.5, e.g. add 4 μl of sample to 26 μl of Cas13a/sgRNA duplex. Incubate the triple complex for 10-15 mins at 37° C. for collateral activity.

Step 4: Pour the sample/Cas13a/sgRNA triple complex on the FeCN6-3 electrode, measure the change in signal.

Electrochemical impedance spectroscopy and/or cyclic voltammetry are utilized to detect one or more compounds on the sensor surface. One or more electrochemical techniques may be used to detect the one or more compounds, such as differential pulse voltammertry (DPV) or square wave voltammetry (SWV).

The embodiments disclosed herein utilize sgRNA targeting effectors to provide a robust CRISPR-based diagnostic with attomolar sensitivity. Embodiments disclosed herein can detect both DNA and RNA with comparable levels of sensitivity and can differentiate targets from non-targets based on single base pair differences. Moreover, the embodiments disclosed herein can be prepared in incubated format for convenient distribution and point-of-care (POC) applications. Such embodiments are useful in multiple scenarios in human health including, for example, viral detection, bacterial strain typing, sensitive genotyping, and detection of disease-associated cell free RNA.

In some aspects, the change of electrical signal before and after the introduction of the target (signal gain) depends on the quantity of hairpin surface probes opened by the target nucleic acid. Various studies have demonstrated an enhancement of signal gain through tuning different factors, such as voltammetric parameters, surface passivation condition and probe density, and stem composition of the hairpin probe in order to achieve an ideal conformational change for a satisfied sensing performance.

The CRISPR/Cas system provides a powerful gene-editing tool that performs high-specificity recognition activity through the Cas enzyme-sgRNA duplex. The following cleavage activity on the target DNA through the Cas enzyme provides the occupancy on the genome to allow the cell to operate its own repair mechanism, achieving genome editing. The target recognition induced cleavage activity of CRISPR may be used for an electrochemical sensor. The nucleic acid target and its complement (opened hairpin containing the electrochemical tag) can be cleaved by Cas enzyme, therefore releasing the electrochemical tag from the sensor surface. This process leads to an absolute loss of the signal, therefore surpassing the detection limit. The ultimate signal gain would be the elimination of the electrochemical signal in the presence of the target, indicating that the removal of the electrochemical tag upon the target recognition is desirable. CRISPR Cas systems provide the opportunity to produce an absolute signal gain through the programmable and specific RNA guided cleavage activity.

The embodiments disclosed herein demonstrate that the CRISPR/Cas-based platform can detect COVID19 virus in clinical samples and SARS family pools with some amount of copies sensitivity and show it can detect COVID19 directly from saliva in <30 minutes. To diagnose infectious microbes around the world, there is a need for assays that are fast, sensitive, low-cost, user-friendly, and rapidly adaptable to detect newly identified agents. Assays were also developed to simultaneously detect 4 influenza variants and to differentiate between SARS virus serotypes 1-4 using region specific variants. Finally, this demonstrates that the methods and devices described herein can detect viruses directly from bodily fluids, distinguish multiple pathogenic viruses, and can be rapidly be updated to identify mutations responsible for clinically important phenotypes.

A biosensing strategy that can discriminate point mutation would be beneficial for genetic disorder analysis. However, current electrochemical mutation detection strategies mostly depend on the variation of RNA medicated charge transport process, which can only indicate mutations through signal comparison between wild-type and mutated sequences at same concentration of the targets. It was hypothesized that the incorporation of target complementarity dependent CRISPR cleavage activity into the electrochemical sensor would allow a second-time recognition activity with a corresponding complementarity dependent cleavage signal. This may be combined with a biosensor conformational change induced signal to indicate the presence of one or more mutations without knowing the target concentration, therefore surpassing the detection accuracy and providing a unique strategy for mutation analysis

### Additional Embodiments

In one embodiment described herein, a nucleic acid detection system comprises a CRISPR system comprising an effector protein and one or more guide RNAs designed to bind to corresponding target molecules; and an RNA-based immobilized construct; wherein the target molecules comprise one or more viral target molecules.

In another embodiment described herein, a polypeptide detection system comprises a CRISPR system comprising an effector protein and one or more guide RNAs designed to bind to a trigger RNA; an RNA-based masking construct; and one or more detection aptamers comprising a masked RNA polymerase promoter binding site or a masked primer binding site.

In one embodiment described herein, a method for detecting viruses in samples comprises distributing a sample or set of samples into one or more individual discrete volumes, the individual discrete volumes comprising a CRISPR system described herein; incubating the sample or set of samples under conditions sufficient to allow binding of the one or more guide RNAs to one or more target molecules; activating the CRISPR effector protein via binding of the one or more guide RNAs to the one or more target molecules, wherein activating the CRISPR effector protein results in modification of the RNA-based masking construct such that a detectable positive signal is generated; and detecting the detectable positive signal, wherein detection of the detectable positive signal indicates a presence of one or more viruses in the sample.

In another embodiment described herein, a method of detecting viruses comprises exposing a CRISPR system described herein to a sample; activating the RNA protein via binding of the one or more guide RNAs to the one or more microbe-specific target RNAs or one or more trigger RNAs such that a detectable positive signal is produced; and detecting the detectable positive signal, wherein detection of the detectable positive signal indicates a presence of one or more viruses in the sample.

In some embodiments, a sample comprises two or more viruses, and the methods described herein may distinguish between the two or more viruses. In some aspects, the sample is a biological sample (e.g., the sample may be an infected sample obtained from a tissue sample, saliva, blood, plasma, sera, stool, urine, sputum, mucous, lymph, synovial fluid, cerebrospinal fluid, ascites, pleural effusion, seroma, pus, or swab of skin or a mucosal membrane surface). In some embodiments, the biological samples are crude samples and/or the one or more target molecules are not purified from the sample prior to application of the method. In some embodiments, the guide RNAs detect single nucleotide variants of the one or more viruses. In some embodiments, the guide RNAs of the one or more CRISPR systems comprise a pan-viral guide RNA set that detects each virus and/or viral strain in a set of viruses. In some embodiments, the guide RNAs are derived using a set cover approach.

In some embodiments, the CRISPR system described herein is located on a substrate, and the substrate is exposed to the sample. In some embodiments, the marker compound is an immobilized-on screen printed gold electrode. In some aspects, one or more CRISPR systems are applied to multiple discrete locations on the substrate. In some embodiments, the different CRISPR systems detect a different microbe at each location. In some embodiments, the substrate is exposed to the sample passively, by temporarily immersing the substrate in a fluid to be sampled, by applying a fluid to be tested to the substrate, or by contacting a surface to be tested with the substrate.

In one embodiment described herein, a method for monitoring viral disease outbreaks and/or evolution comprises exposing a CRISPR system described herein to a sample, activating the RNA effector protein via binding of the one or more guide RNAs to the one or more target sequences comprising non-synonymous viral mutations such that a detectable positive signal is produced; and detecting the detectable positive signal, wherein detection of the detectable positive signal indicates a type of viral strain that is present in the sample. In some aspects, exposing the CRISPR system comprises locating one or more CRISPR systems within one or more individual discrete volumes and adding a sample or sample aliquot to the one or more individual discrete volumes.

In another embodiment described herein, a method for screening samples for viral antigens and/or viral specific antibodies comprises exposing a CRISPR system described herein to a sample, wherein the one or more aptamers bind to one or more viral antigens or one or more viral-specific antibodies, and wherein the aptamers encode a barcode that identifies the one or more viral antigens or one or more viral specific antibodies that the one or more aptamers bind to, and wherein the guide RNAs are designed to detect the barcode; activating the RNA effector protein via binding of the one or more guide RNAs to the one or more microbe-specific target RNAs or one or more trigger RNAs such that a detectable positive signal is produced; and detecting the detectable positive signal, wherein detection of the detectable positive signal indicates a presence of one or more viral antigens or one/or more viral specific antibodies in the sample.

In some embodiments, the virus is a DNA virus, e.g., a Myoviridae, Podoviridae, Siphoviridae, Alloherpesviridae, Herpesviridae (including human herpes virus, and Varicella Zorter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus, or combination thereof.

In some embodiments, the viral infection is caused by a double-stranded RNA virus, a positive sense RNA virus, a negative sense RNA virus, a retrovirus, or a combination thereof. In some embodiments, the virus is a Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In some embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In another embodiment described herein, a method for detecting one or more microbes in a sample, comprises contacting a sample with a nucleic acid detection system (e.g., a CRISPR system) described herein; and applying said contacted sample to an electrochemical biosensor strategy.

In some embodiments, the nucleic acid detection system comprises an RNA-based immobilized construct comprising a first and a second molecule and said electrochemical sensing strategy comprises detecting said first and second molecule, preferably at discrete detection sites on an immobilized biosensor. In some embodiments, the first molecule or second molecule is detected by binding to an antibody recognizing said first or second molecule and detecting said bound molecule, preferably with sandwich antibodies. In some embodiments, the combination of an electrochemistry-based biosensor and CRISPR, delivers an integrated sensor and actuator system as a powerful biosensing platform, and uncleaved RNA-based masking construct is bound by said first antibody if the target nucleic acid is not present in said sample, and cleaved RNA-based immobilized construct is bound both by said first antibody or said second antibody if the target nucleic acid is present in said sample.

In one embodiment described herein, a method of detecting a virus comprises obtaining a sample from a subject, wherein the sample is blood or saliva; electrochemical sensing of the sample RNA; combining the sample with an effector protein, one or more guide RNAs designed to bind to corresponding virus-specific target molecules, and an RNA-based masking construct, activating the RNA effector protein via binding of the one or more guide RNAs to the one or more virus-specific target RNAs, wherein activating the CRISPR effector protein results in modification of the RNA-based masking construct such that a detectable positive signal is produced; and detecting the signal, wherein detection of the signal indicates the presence of the virus; and wherein the method does not include a step of extracting RNA from the sample.

In some embodiments, the biosensing step is of a duration less than 1 minute. In some embodiments, the detection step is of a duration less than 30 minutes. In some embodiments, the volume of sample required for detection is 20 [symbol] . In some embodiments, the volume of sample required in an Eppendorf tube incubation required 5 min using Lysis buffer then neutralized with PBS/Potassium acetate/any neutralizing solution. In some embodiments, the prepared Cas13a/sgRNA duplex takes 5-10 minutes with incubation at 25° C. In some embodiments, the sample prepared with biological sample (blood or saliva)/Cas13a/sgRNA triplex takes 10-15 minute for collateral activity. In some embodiments, the virus is Coronaviridae virus. In some embodiments, the sample comprises two or more viruses and wherein the method distinguishes between the two or more viruses. In some embodiments, the sample/Cas13a/sgRNA triple complex is poured or otherwise deposited on the FeCN6⁻³ electrode, and the change in signal is measured. In some embodiments, the method further comprises a nuclease inactivation step and a viral inactivation step prior to electrochemical biosensing the sample RNA.

In still another embodiment described herein, a nucleic acid detection system comprises a CRISPR system comprising an effector protein and one or more guide RNAs designed to bind to corresponding target molecules; an RNA-based masking construct; and optionally, nucleic acid reagents to detect target RNA molecules in a sample. In another aspect, the embodiments provide a polypeptide detection system comprising: a CRISPR (Cas13a) system comprising an effector protein and one or more guide RNAs designed to bind a trigger RNA, a sgRNA-based masking construct; and one or more detection aptamers comprising a masked RNA polymerase promoter binding site or a masked trimer binding site.

In other embodiments describe herein, a diagnostic device comprises one or more individual discrete volumes, each individual discrete volume comprising a CRISPR effector protein, one or more guide RNAs designed to bind to a trigger RNA, one or more detection aptamers comprising a masked RNA polymerase promoter binding site or a masked primer binding site, and optionally further comprising nucleic acid reagents.

In another embodiment described herein, a sensor comprises a counter electrode; multiple working electrodes which include immobilized biological molecules; a reference electrode; and a support on which the electrodes are disposed.

What is claimed is:

1. A handheld portable device for detecting and identifying one or more nucleic acids in a fluid sample of a subject comprising:
a sensor cartridge comprising:
a sample well; and
one or more disposable biosensors, wherein a biosensor comprises
a Cas13a protein or a Cas12a protein,
a methylene blue tagged RNA or DNA reporter sequence which is thiol group functionalized at one terminal, and
one or more guide Ribonucleic acid (gRNA) gRNA for severe acute respiratory syndrome coronavirus 2 (SARS-CoV-2) N RNA, SARS-CoV-2 S RNA, or ORF1ab gene region of SARS-CoV-2; and
a hub station comprising:
an electrochemical reader, the electrochemical reader analyzing the one or more biosensors to provide biosensor data as multiple channel signals in real-time when the sensor cartridge is inserted into the electrochemical reader;
a communication apparatus; and
a battery,
wherein the hub station receives the sensor cartridge.

2. The device of claim 1, further comprising a processing apparatus electronically or wirelessly connected to the communication apparatus, wherein the processing apparatus receives data from the communication apparatus and processes the data to identify a disease based on the one or more nucleic acids.

3. The device of claim 2, wherein the processing apparatus transmits the identity of the disease detected in the sample from the processing apparatus to a display.

4. The device of claim 1, wherein the Cas13a protein or Cas12a protein, the thiol functionalized reporter sequence, and the one or more gRNA are immobilized on screen printed gold electrodes.

5. The device of claim 1, wherein the biosensor further comprises an electrode assembly comprising one counter electrode, four to eight working electrodes, and one reference electrode.

6. The device of claim 1, wherein the biosensor further comprises an amplification agent.

7. The device of claim 6, wherein the amplification agent is selected from the group consisting of T4 RNA ligase, DNA polymerase, RNA polymerase, and t4 polynucleotide kinase (pnk).

8. The device of claim 1, wherein the biosensor further comprises a binding agent.

9. The device of claim 8, wherein the binding agent is selected from the group consisting of carbodiimide compounds and carboxyl-reactive crosslinker reactive groups.

10. A method for detecting and identifying one or more nucleic acid compounds in a sample of a subject comprising:
receiving a collected sample in a sensor cartridge of a portable device, wherein the sensor cartridge comprises one or more biosensors comprising a Cas13a protein or a Cas12a protein, a methylene blue tagged RNA or DNA reporter sequence which is thiol group functionalized at one terminal, and one or more guide Ribonucleic acid (gRNA) for severe acute respiratory syndrome coronavirus 2 (SARS-CoV-2) N RNA, SARS-CoV-2 S RNA, or ORF1ab gene region of SARS-CoV-2;
inserting the sensor cartridge into a hub station of the portable device, the hub station including an electrochemical reader;
the electrochemical reader analyzing the one or more biosensors using square wave voltammetry to provide biosensor data as multiple channel signals in real-time;
communicating the biosensor data to a processing apparatus via a communication apparatus;
detecting one or more SARS-CoV-2 nucleic acids in the sample;
identifying the sample as being positive for COVID19 to provide a COVID 19 diagnosis; and
transmitting the COVID19 diagnosis to a user of the device.

11. The method of claim 10, wherein the sample is a blood or saliva sample.

12. The method of claim 10, wherein the diagnosis of COVID19 occurs within 2 minutes of receiving the sample.
